(19) United States
(12) Patent Application Publication
(10) Pub. No.: US 2010/0256331 A1
(43) Pub. Date: Oct. 7, 2010

(54) 68Ga-Labelling of a Free and Macromolecule Conjugated Macrocyclic Chelator at Ambient Temperature

(75) Inventors: Irina Velikyan, Uppsala (SE); Bengt Langstrom, Uppsala (SE)

Correspondence Address: GE HEALTHCARE, INC., IP DEPARTMENT 101 CARNEGIE CENTER, PRINCETON, NJ, 08540-6231, US

(73) Assignee: GE Healthcare Limited, Buckinghamshire (GB)

(21) Appl. No.: 12/377,887

(22) PCT Filed: Aug. 29, 2007
(86) PCT No.: PCT/IB07/02499

§ 371 Date: May 24, 2010

Related U.S. Application Data
(60) Provisional application No. 60/823,797, filed on Aug. 29, 2006.

(51) Int. Cl. C07K 2/00; C07F 5/00

(52) U.S. Cl. 530/322; 534/10; 530/300

(57) ABSTRACT

The present invention relates to a method of producing radiolabelled gallium complexes at ambient temperature that could be used as diagnostic agents, e.g. for positron emission tomography (PET) imaging.

### FIELD OF THE INVENTION

The present invention relates to a method of producing radiolabelled gallium complexes at ambient temperature. The complexes could be used as diagnostic agents, e.g. for positron emission tomography (PET) imaging.

### BACKGROUND OF THE INVENTION

PET imaging is a tomographic nuclear imaging technique that uses radioactive tracer molecules that emit positrons. When a positron meets an electron, the both are annihilated and the result is a release of energy in form of gamma rays, which are detected by the PET scanner. By employing natural substances that are used by the body as tracer molecules, PET does not only provide information about structures in the body but also information about the physiological function of the body or certain areas therein. A common tracer molecule is for instance 2-fluoro-2-deoxy-D-glucose (FDG), which is similar to naturally occurring glucose, with the addition of a ¹⁸F-atom. Gamma radiation produced from said positron-emitting fluorine is detected by the PET scanner and shows the metabolism of FDG in certain areas or tissues of the body, e.g. in the brain or the heart. The choice of tracer molecule depends on what is being scanned. Generally, a tracer is chosen that will accumulate in the area of interest, or be selectively taken up by a certain type of tissue, e.g. cancer cells. Scanning consists of either a dynamic series or a static image obtained after an interval during which the radioactive tracer molecule enters the biochemical process of interest. The scanner detects the spatial and temporal distribution of the tracer molecule. PET also is a quantitative imaging method allowing the measurement of regional concentrations of the radioactive tracer molecule.

Commonly used radionuclides in PET tracers are ¹¹C, ¹⁸F, ¹⁵O, ¹³N or ⁷⁶Br. Recently, new PET tracers were produced that are based on radiolabelled metal complexes comprising a bifunctional chelating agent and a radiometal. Bifunctional chelating agents are chelating agents that coordinate to a metal ion and are linked to a targeting vector that will bind to a target site in the patient's body. Such a targeting vector may be a peptide that binds to a certain receptor, probably associated with a certain area in the body or with a certain disease. A targeting vector may also be an oligonucleotide specific for e.g. an activated oncogene and thus aimed for tumour localisation. The advantage of such complexes is that the bifunctional chelating agents may be labelled with a variety of radiometals like, for instance, ⁶⁸Ga, ²¹³Bi or ⁸⁶Y. In this way, radiolabelled complexes with special properties may be “tailored” for certain applications.

⁶⁸Ga is of special interest for the production of Ga-radiolabelled metal complexes used as tracer molecules in PET imaging. ⁶⁸Ga is obtained from a ⁶⁸Ge/⁶⁸Ga generator, which means that no cyclotron is required. ⁶⁸Ga decays to 89% by positron emission of 2.92 MeV and its 68 min half-life is sufficient to follow many biochemical processes in vivo without unnecessary radiation. With its oxidation state of +III, ⁶⁸Ga forms stable complexes with various types of chelating agents and ⁶⁸Ga tracers have been used for brain, renal, bone, blood pool, lung and tumour imaging.

J. Schumacher et al., Cancer Res. 61, 2001, 3712-3717 describe the synthesis of ⁶⁸Ga-N,N′[2-hydroxy-5-(ethylene-β-carboxy)benzyl]ethylenediamine-N,N′-diacetic acid (⁶⁸Ga-HBED-CC). ⁶⁸Ga obtained from a ⁶⁸Ge/⁶⁸Ga generator and Ga³⁺ carrier are reacted with the chelating agent HBED-CC in acetate buffer for 15 min at 95° C. Uncomplexed ⁶⁸Ga is separated from the complex using a cation exchange column. The overall preparation is reported to take 70 min. A disadvantage of this method is that the overall preparation time of the radiolabelled complex is very long. Due to the addition of “cold” Ga³⁺ carrier, the specific activity of the reaction is low. Moreover, the radiolabelled complex had to be purified after the complex formation reaction.

Ö. Ugur et al., Nucl. Med. Biol. 29, 2002, 147-157 describe the synthesis of the ⁶⁸Ga labelled somatostatin analogue DOTA-DPhe¹-Tyr³-octreotide (DOTATOC). The compound is prepared by reacting ⁶⁸GaCl₃ obtained from a ⁶⁸Ge/⁶⁸Ga generator with the chelating agent DOTATOC for 15 min at 100° C. A disadvantage of this method is that the reaction mixture had to be heated at relatively high temperatures. The DOTA chelating agent was functionalised with a peptide targeting vector and peptides and proteins are substances, which are known to be sensitive to heat. Thus, with the method described there is a risk that heat sensitive targeting vectors are destroyed during complex formation. A further disadvantage is that the complex had to be purified by HPLC before it could be used for animal studies.

U.S. Pat. No. 5,070,346 discloses ⁶⁸Ga-labelled complexes of the chelating agent tetraethylcyclohexyl-bis-aminoethanethiol (BAT-TECH). The complexes are synthesised by reacting ⁶⁸GaCl₃ obtained from a ⁶⁸Ge/⁶⁸Ga generator with BAT-TECH at 75° C. for 15 min and subsequent filtration. The preparation of the complex was accomplished in 40 min. Due to the high reaction temperature; this method would not be suitable for bifunctional chelating agents comprising a heat sensitive targeting vector, for instance a peptide or a protein. A further disadvantage is the long reaction time of the complex formation reaction.

We have recently developed a novel method of using microwave activation to substantially improve the efficiency and reproducibility of the ⁶⁸Ga-chelating agent complex formation. In WO 2004/089425, we disclosed a microwave activation method which provides shorter reaction time, increased selectivity of radiolabeling reaction and increased radiochemical yield. While microwave activation has a positive effect on radiolabelling with all Ga-radioisotopes, namely with ⁶⁶Ga, ⁶⁷Ga and ⁶⁸Ga, due to high reaction temperature, this method is still not optimal for chelating agents comprising macromolecules such as large peptides, proteins, antibodies, antibody fragments, glycoproteins or oligonucleotides.

In view of the foregoing, there is a need for a fast and easier method for the synthesis of ⁶⁸Ga-labelled complexes at ambient temperature, which could be used as tracer molecules for PET imaging.

### SUMMARY OF THE INVENTION

The invention thus provides a method of producing a radiolabelled gallium complex by reacting a Ga³⁺ radioisotope with a chelating agent characterised in that the reaction is carried out at ambient temperature.

In a preferred embodiment of the instant invention, the Ga³⁺ radioisotope is ⁶⁸Ga³⁺.

In another preferred embodiment of the instant invention, the chelating agent is a macrocyclic chelating agent, preferably NOTA. The chelating agent can be either in a free form, or coupled with a targeting vector.

In a further preferred embodiment of the invention, the chelating agent is a bifunctional chelating agent, preferably NOTA, comprising a targeting vector selected from the group comprising proteins, glycoproteins, lipoproteins, polypeptides, glycopolypeptides, lipopolypeptides, peptides, glycopeptides, lipopeptides, carbohydrates, nucleic acids, oligonucleotides or a part, a fragment, a derivative or a complex of the aforesaid compounds and small organic molecules.

### BRIEF DESCRIPTION OF THE FIGURES

FIG. 1 shows the time course of ⁶⁸Ga complexation reaction conducted using 1 mL peak fraction of the generator eluate at ambient temperature for varied amount of NODAGATATE.

FIG. 2 shows the time course of ⁶⁸Ga-NOTA formation reaction conducted using 1 mL peak fraction of the generator eluate at ambient temperature.

### DETAILED DESCRIPTION OF THE INVENTION

The instant invention provides a method of producing a radiolabelled gallium complex by reacting a Ga³⁺ radioisotope with a chelating agent characterised in that the reaction is carried out at ambient temperature. One advantage of the instant invention is to simplify even further the PET tracer preparation and allow for “shoot and shake” labelling analogous to one carried out with the SPECT isotope ^{99m}Tc. Another advantage for this fast ⁶⁸Ga-labelling reaction at ambient temperature is that it becomes a valuable tool when producing temperature sensitive macromolecular tracers.

Suitable Ga³⁺ radioisotopes according to the invention are ⁶⁶Ga³⁺, ⁶⁷Ga³⁺ and ⁶⁸Ga³⁺, preferably ⁶⁶Ga³⁺ and ⁶⁸Ga³⁺ and particularly preferably ⁶⁸Ga³⁺. ⁶⁶Ga³⁺ and ⁶⁸Ga³⁺ are particularly suitable for the production of radiolabelled complexes useful in PET imaging whereas ⁶⁷Ga³⁺ is particularly suitable for the production of radiolabelled complexes useful in single photon emission computerised tomography (SPECT).

⁶⁶Ga³⁺ is obtainable by cyclotron production by irradiation of elemental zinc targets. To minimise the amounts of ⁶⁷Ga production, the target thickness is preferably maintained such that the degraded proton energy is above 8 MeV, and irradiation time is kept short, e.g. <4 hrs. The chemical separation may be achieved using solvent-solvent extraction techniques using isopropyl ether and HCl as described in L. C. Brown, Int. J. Appl. Radiat. Isot. 22, 1971, 710-713. ⁶⁶Ga has a relatively long half-life of 9.5 h and the most abundant positron emitted has a uniquely high energy of 4.2 MeV.

⁶⁷Ga³⁺ is obtainable by cyclotron production and ⁶⁷GaCl₃ obtained by cyclotron production is a commercially available compound. The half-life of ⁶⁷Ga is 78 h.

⁶⁸Ga is obtainable from a ⁶⁸Ge/⁶⁸Ga generator. Such generators are known in the art and for instance described by C. Loc'h et al, J. Nucl. Med. 21, 1980, 171-173. Generally, ⁶⁸Ge is loaded onto a column consisting of an organic resin or an inorganic metal oxide like tin dioxide, aluminium dioxide or titanium dioxide. ⁶⁸Ga is eluted from the column with aqueous HCl, yielding ⁶⁸GaCl₃. ⁶⁸Ga³⁺ is particularly preferred in the method according to the invention as its production does not require a cyclotron and its 68 min half-life is sufficient to follow many biochemical processes in vivo by PET imaging without long radiation.

Preferred chelating agents for use in the method of the invention are those which present the Ga³⁺ radioisotopes in a physiologically tolerable form. Further preferred chelating agents are those that form complexes with Ga³⁺ radioisotopes that are stable for the time needed for diagnostic investigations using the radiolabelled complexes.

Macrocyclic chelating agents are preferably used in the method of the invention. In a preferred embodiment, these macrocyclic chelating agents comprise at least one hard donor atom such as oxygen and/or nitrogen like in polyaza- and polyoxomacrocycles.

Particularly preferred macrocyclic chelating agents comprise functional groups such as carboxyl groups or amine groups which are not essential for coordinating to Ga³⁺ and thus may be used to couple other molecules, e.g. targeting vectors, to the chelating agent. The chelating agent can be in a free form, or coupled with a targeting vector. A preferred example of such macrocyclic chelating agent comprising functional group of NOTA.

The general structure of NOTA and its derivative chelators is shown below and consists of three macrocyclic amine groups and three carboxylic groups for coordination to Ga(III) and an additional functional group (Y₁) such that the chelate can be conjugated to a vector, preferably alkylamine, alkylsulphide, alkoxy, alkyl carboxylate, arylamine, aryl sulphide or α-haloacetyl; Y₂ and Y₃ can be H or contain one or more functional moieties that would on the one hand improve the complexation depending on a particular metal cation and on the other hand change the overall charge and hydrophilicity of the complex in order to modify the pharmacokinetics and blood clearance rates, preferably alkylamine, alkoxy, alkyl carboxylate, phenol, hydroxamate, aryl sulphide, alkyl.

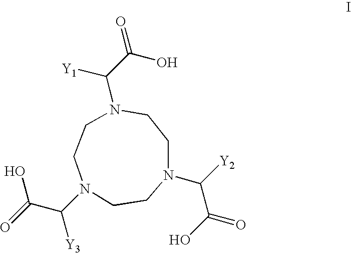

In a further preferred embodiment, bifunctional chelating agents are used in the method according to the invention. “Bifunctional chelating agent” in the context of the invention means chelating agents that are linked to a targeting vector. Suitable targeting vectors for bifunctional chelating agents useful in the method according to the invention are chemical or biological moieties, which bind to target sites in a patient's body, when the radiolabelled gallium complexes comprising said targeting vectors have been administered to the patient's body. Suitable targeting vectors for bifunctional chelating agents useful in the method according to the invention are proteins, glycoproteins, lipoproteins, polypeptides like antibodies or antibody fragments, glycopolypeptides, lipopolypeptides, peptides, like RGD binding peptides, glycopeptides, lipopeptides, carbohydrates, nucleic acids e.g. DNA, RNA, oligonucleotides like antisense oligonucleotides or a part, a fragment, a derivative or a complex of the aforesaid compounds, or any other chemical compound of interest, such as small organic molecules.

In a particularly preferred embodiment, macrocyclic bifunctional chelating agents are used in the method according to the invention. Preferred macrocyclic bifunctional chelating agent is NOTA linked to a targeting vector, preferably to a targeting vector selected from the group comprising proteins, glycoproteins, lipoproteins, polypeptides, glycopolypeptides, lipopolypeptides, peptides, glycopeptides, lipopeptides carbohydrates, nucleic acids, oligonucleotides or a part, a fragment, a derivative or a complex of the aforesaid compounds and small organic molecules; particularly preferably to a targeting vector selected from the group consisting of peptides and oligonucleotides.

The targeting vector can be linked to the chelating agent via a linker group or via a spacer molecule. Examples of linker groups are disulfides, ester or amides, examples of spacer molecules are chain-like molecules, e.g. lysin or hexylamine or short peptide-based spacers. In a preferred embodiment, the linkage between the targeting vector and the chelating agent part of radiolabelled gallium complex is as such that the targeting vector can interact with its target in the body without being blocked or hindered by the presence of the radiolabelled gallium complex. A general structure of NOTA-based bifunctional chelating agent linked to a targeting vector is shown below:

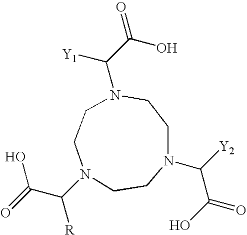

wherein Y₁ and Y₂ as defined above and R is the targeting vector comprising proteins, glycoproteins, lipoproteins, polypeptides, glycopolypeptides, lipopolypeptides, peptides, glycopeptides, lipopeptides carbohydrates, nucleic acids, oligonucleotides or a part, a fragment, a derivative or a complex of the aforesaid compounds and small organic molecules; particularly preferably to a targeting vector selected from the group consisting of peptides and oligonucleotides.

The labelling reaction according to the instant invention comprises the following steps: obtaining ⁶⁸Ga³⁺ from a 68Ge/68Ga generator in a buffered solution; conjugating a targeting vector with a suitable chelating agent, preferably NOTA to form bioconjugate; adding the bioconjugate to the ⁶⁸Ga³⁺ buffered solution; incubate the reaction mixture at ambient temperature to give radiolabelled gallium complex, namely, ⁶⁸Ga-chelating agent-targeting vector.

The step of obtaining ⁶⁸Ga³⁺ from a 68Ge/68Ga generator in a buffered solution is described in the sections below. In a preferred embodiment, the buffered solution is in HEPES or sodium acetate.

An example of biojugation is provided in one of the examples below. Incubation period will be the reaction time of the reaction mixture, which will be less than ten minutes.

In a preferred embodiment, the invention provides a method of producing a ⁶⁸Ga radiolabelled PET imaging tracer by reacting ⁶⁸Ga³⁺ with a macrocyclic bifunctional chelating agent, characterised in that the reaction is carried out at ambient temperature. Ambient temperature is preferably from 20° C. to 25° C.

In a particularly preferred embodiment of the method described in the last preceding paragraph, the incubation step is carried out in less than ten minutes.

If ⁶⁸Ga³⁺ is used in the method according to the invention, the ⁶⁸Ga³⁺ is preferably obtained by contacting the eluate form a ⁶⁸Ge/⁶⁸Ga generator with an anion exchanger and eluting ⁶⁸Ga³⁺ from said anion exchanger. In a preferred embodiment, the anion exchanger is an anion exchanger comprising HCO₃⁻ as counterions.

The use of anion exchangers to treat ⁶⁸Ga eluate obtained from a ⁶⁸Ge/⁶⁸Ga generator is described by J. Schuhmacher et al. Int. J. appl. Radiat. Isotopes 32, 1981, 31-36. A Bio-Rad AG 1×8 anion exchanger was used for treating the 4.5 NHCl ⁶⁸Ga eluate obtained from a ⁶⁸Ge/⁶⁸Ga generator in order to decrease the amount of ⁶⁸Ge present in the eluate.

It has now been found that the use of anion exchangers comprising HCO₃⁻ as counterions is particularly suitable for the purification and concentration of the generator eluate. Not only the amount of ⁶⁸Ge present in the eluate could be reduced but also the amount of so-called pseudo carriers, i.e. other metal cations like Fe³⁺, Al³⁺, Cu²⁺, Zn²⁺, and In³⁺, that are eluted together with the ⁶⁸Ga³⁺ from the generator. As these pseudo carriers compete with ⁶⁸Ga³⁺ in the subsequent complex formation reaction, it is especially favourable to reduce the amount of those cations as much as possible before the labelling reaction. A further advantage of the anion-exchange purification step is that the concentration of ⁶⁸Ga³⁺, which is in the picomolar to nanomolar range after the elution, can be increased up to a nanomolar to micromolar level. Hence, it is possible to reduce the amount of chelating agent in a subsequent complex formation reaction, which considerably increases the specific radioactivity. This result is important for the production of ⁶⁸Ga-radiolabelled PET tracers that comprise a bifunctional chelating agent; i.e. a chelating agent linked to a targeting vector, as the increase in specific radioactivity enables the reduction in amount of such tracers when used in a patient.

Hence, another preferred embodiment of the method according to the invention is a method of producing a ⁶⁸Ga-radiolabelled complex by reacting ⁶⁸Ga³⁺ with a chelating agent using microwave activation, wherein the ⁶⁸Ga³⁺ is obtained by contacting the eluate form a ⁶⁸Ge/⁶⁸Ga generator with an anion exchanger, preferably with an anion exchanger comprising HCO₃⁻ as counterions, and eluting ⁶⁸Ga³⁺ from said anion exchanger.

⁶⁸Ge/⁶⁸Ga generators are known in the art, see for instance C. Loc'h et al, J. Nucl. Med. 21, 1980, 171-173 or J. Schuhmacher et al. Int. J. appl. Radiat. Isotopes 32, 1981, 31-36. ⁶⁸Ge may be obtained by cyclotron production by irradiation of, for instance Ga₂(SO₄)₃ with 20 MeV protons. It is also commercially available, e.g. as ⁶⁸Ge in 0.5 M HCl. Generally, ⁶⁸Ge is loaded onto a column consisting of organic resin or an inorganic metal oxide like tin dioxide, aluminium dioxide or titanium dioxide. ⁶⁸Ga is eluted from the column with aqueous HCl yielding ⁶⁸GaCl₃.

Suitable columns for ⁶⁸Ge/⁶⁸Ga generators consist of inorganic oxides like aluminium dioxide, titanium dioxide or tin dioxide or organic resins like resins comprising phenolic hydroxyl groups (U.S. Pat. No. 4,264,468) or pyrogallol (J. Schuhmacher et al., Int. J. appl. Radiat. Isotopes 32, 1981, 31-36). In a preferred embodiment, a ⁶⁸Ge/⁶⁸Ga generator comprising a column comprising titanium dioxide is used in the method according to the invention.

The concentration of the aqueous HCl used to elute the ⁶⁸Ga from the ⁶⁸Ge/⁶⁸Ga generator column depends on the column material. Suitably 0.05 to 5 M HCl is used for elution of ⁶⁸Ga. In a preferred embodiment, the eluate is obtained from a ⁶⁸Ge/⁶⁸Ga generator comprising a column comprising titanium dioxide and ⁶⁸Ga is eluted using 0.05 to 0.1 M HCl, preferably about 0.1 M HCl.

In a preferred embodiment of the method according to the invention, a strong anion exchanger comprising HCO₃⁻ as counterions, preferably a strong anion exchanger comprising HCO₃⁻ as counterions, is used. In a further preferred embodiment, this anion exchanger comprises quaternary amine functional groups. In another further preferred embodiment, this anion exchanger is a strong anion exchange resin based on polystyrene-divinylbenzene. In a particularly preferred embodiment, the anion exchanger used in the method according to the invention is a strong anion exchange resin comprising HCO₃⁻ as counterions, quaternary amine functional groups and the resin is based on polystyrene-divinylbenzene.

Suitably, water is used to elute the ⁶⁸Ga from the anion exchanger in the method according to the invention.

The ⁶⁸Ga elute obtained according to the instant invention is buffered in HEPES or sodium acetate for labelling reactions.

### EXAMPLES

The invention is further described in the following examples which are in no way intended to limit the scope of the invention.

### Example 1

### ⁶⁸Ga -Radiolabelling of NODAGA-TATE

### 1a) Materials

HEPES (4-(2-Hydroxyethyl) piperazine-1-ethanesulfonic acid), sodium acetate and double distilled hydrochloric acid (Riedel de Haën) were obtained from Sigma-Aldrich Sweden (Stockholm, Sweden). Sodium dihydrogen phosphate, di-sodium hydrogen phosphate and trifluoroacetic acid (TFA) were obtained from Merck (Darmstadt, Germany). The purchased chemicals were used without further purification. Deionised water (18.2 MΩ), produced with a Purelab Maxima Elga system (Bucks, UK) was used in all reactions.

### 1b) ⁶⁸Ga Production

⁶⁸Ga (T_{1/2}=68 min, β⁺=89% and EC=11%) was available from a ⁶⁸Ge/⁶⁸Ga-generator-system (Cyclotron Co., Ltd, Obninsk, Russia) where ⁶⁸Ge (T_{1/2}=270.8 d) was attached to a column of an inorganic matrix based on titanium dioxide. The ⁶⁸Ga was eluted with 6 mL of 0.1 M hydrochloric acid.

### 1c) ⁶⁸Ga-Labelling of NODAGA-TATE

The pH of the ⁶⁸Ge/⁶⁸Ga-generator eluate was adjusted to 3.5-5.0 by adding either HEPES to give finally a 1.0 M solution with regard to HEPES or sodium acetate to give finally a 0.4 M solution with regard to sodium acetate. Then 0.2-20 nmols in 1-20 μL of 1 mM and 1-5 μL of 0.1 mM NODAGA-TATE solution (in water) were added and the reaction mixture was incubated at room temperature. The reaction mixture was analyzed on an HPLC system from Beckman (Fullerton, Calif., USA) consisting of a 126 pump, a 166 UV detector and a radiation detector coupled in series. Data acquisition and handling was performed using the Beckman System Gold Nouveau Chromatography Software Package. The column used was a Vydac RP 300 Å HPLC column (Vydac, USA) with the dimensions 150 mm×4.6 mm, 5 μm particle size. We applied gradient elution with the following parameters: A=10 mM TFA; B=70% acetonitrile (MeCN), 30% H₂O, 10 mM TFA with UV-detection at 220 nm; flow was 1.2 mL/min; 0-2 min isocratic 20% B, 20-90% B linear gradient 8 min, 90-20% B linear gradient 2 min.
To the ⁶⁸Ge/⁶⁸Ga-generator eluate (6 mL) was added 5 mL of 30% HCl resulting in 4.0 M solution (11 mL) which then was passed through an anion exchange cartridge (Chromafix 30-PS-HCO₃, Macharey-Nagel, Germany) at a flow rate of 4 mL/min (linear flow speed 25 cm/min) at ambient temperature. Then the cartridge was dried by sucking filtered air through it, in order to eliminate excess 4 M HCl. The ⁶⁸Ga was then eluted with small fractions of deionized water (50-200 μl) at a flow rate of 0.5 mL/min. Then 3 μL of 10 M NaOH solution were added to the 200 μL of the ⁶⁸Ga preconcentrated eluate containing 92±4% of the initially available ⁶⁸Ga activity and the mixture was transferred to a vial containing 72 mg HEPES powder buffering the solution to 3<pH<3.5. Then 0.2-5 nmols of the NODAGA-TATE were added in 1-5 μL of 1 mM aqueous solution or 2-5 μL of 0.1 mM aqueous solution of the conjugate. The resulting 200±20 μL reaction mixture was incubated at ambient temperature.
The studies on the kinetics of ⁶⁸Ga-labelling of an octapeptide coupled to NOTA chelator showed promising results. The quantitative (>95%) incorporation of ⁶⁸Ga took place at room temperature within short time (<10 min). Primary structure of NODAGATATE, 1,4,7-Tricarboxymethyl-1,4,7-triazacyclononan-1-yl-acetyl-D-Phe-Cys-Tyr-D-Trp-Lys-Thr-Cys-L-Thr (NODAGA-Tyr³-Octreotate) is shown below:

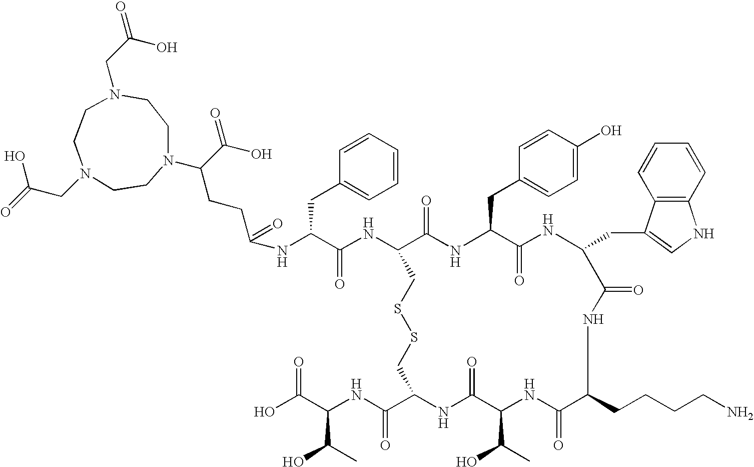

The reaction scheme for the complexation of ⁶⁸Ga with NODAGATATE, where R is Tyr³-Octreotate, is shown as follows:

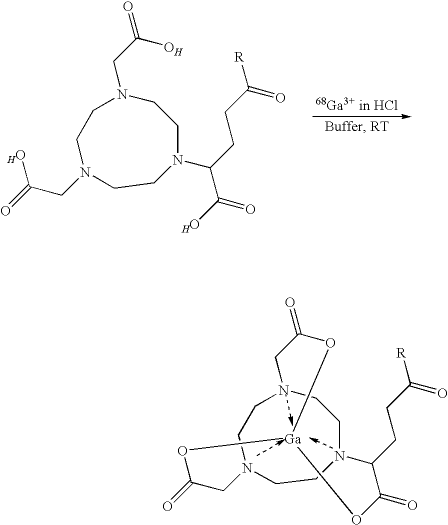

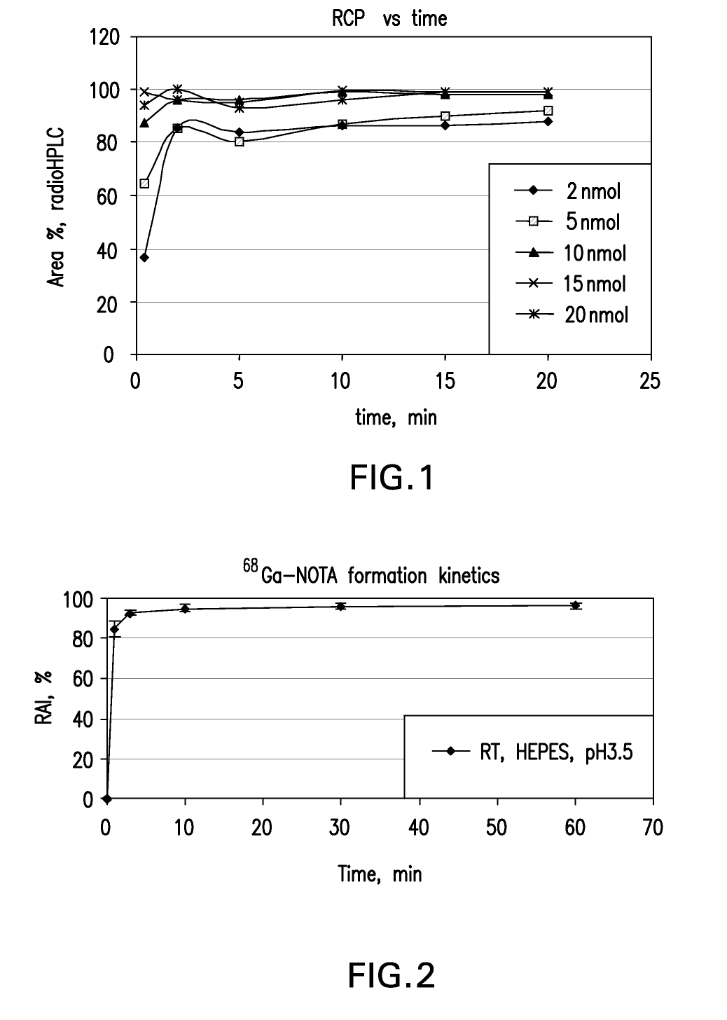

The chelate exhibited fast labelling kinetics with ⁶⁸Ga (FIG. 1) and should have good in vivo stability due to the high thermodynamic stability and extremely slow dissociation.

### Example 2

### ⁶⁸Ga-Labelling of NOTA

HEPES (14 mg) or sodium acetate buffering agents was added to 200 μL, of ⁶⁸Ga and the pH was adjusted with HCl and NaOH to give pH values between two and seven. NOTA (50 nanomoles, synthesized at Grove Centre, GB) was added and the reaction mixture was incubated at room temperature. The reaction mixture was analyzed by Thin Layer Chromatography (TLC) applying the analyte to a polyethyleneimine cellulose plate (PEI-Cellulose F, Merck, Germany) and using 0.4 M NaH₂PO₄ (pH=3.5) as running buffer. Autoradiography was employed to image the TLC strips. A phosphor storage plate (Molecular Dynamics, Amersham Biosciences, the U.K.) was placed on top of the strips. The plate was scanned with Phosphorlmager (PI) SI unit (Molecular Dynamics, Amersham Biosciences, the U.K.) and analysed using ImageQuant 5.1 software. The non-incorporated (free) ⁶⁸Ga stayed at the origin and R_{F} of the ⁶⁸Ga-complex was 0.9. Studies on the kinetics of ⁶⁸Ga-NOTA complex formation resulted in quantitative incorporation (>95%) of ⁶⁸Ga at room temperature within 10 min (FIG. 2).
In both examples the purification of the 68Ga-labelled products was not necessary since the radiochemical purity was >90% and the preparation buffer HEPES/HEPES-Na, (4-(2-Hydroxyethyl) piperazine-1-ethanesulfonic acid) is eligible for human use.

### Example 3

### Conjugation Reactions of DOTA and Macromolecule

1. A macromolecule with amine group was dissolved in 100-300 μl Borax (B₄O₇×10H₂O) and pH was adjusted to 9.5-10 with 5 M NaOH.
2. Then the dissolved macromolecule was added to 50 fold excess of sulfo-NHS (N-Hydroxysulfosuccinimide) ester of NOTA (50 nanomoles, synthesized at Grove Centre, GB) on ice under continuous stirring.
3. The pH was checked and adjusted, if necessary, to 8.5-9 with 5 M NaOH. The mixture was left for overnight at 4° C.
4. On the next day, the mixture was purified in Centricon Centrifugal Filter Unit with Ultracel YM-3 (3 kDa) membrane (Amicon, Danvers, Mass., USA) by 3 successive centrifugations at 7500×g for 2 hours at 4° C. (Beckman J2-MC Centrifuge, Palo Alto, Calif., USA). The volume was adjusted to 2 ml with H₂O before each centrifugation. The retentate liquid was recovered by inverting the filter unit and centrifuging at 610×g for 2 minutes at 4° C.
5. Purity analysis and concentration determination of NOTA-macromolecule were performed using UV-RP-HPLC (Beckman System with a 126 pump, a 166 UV detector and a radiodetector coupled in series, Fullerton, Calif., USA) with a Vydac RP 300 Å column (Vydac, USA) 150×4.6 mm ID, 5 μm; flow 1.5 mL/min, a=20 mM triethylammonium acetate buffer (TEAA); b=100% acetonitrile (MeCN), linear gradient 0-10% b 2-4 min, 10-30% b 4-9 min, 30-50% b 9-15 min; λ=254 nm.
6. The purified product was stored in a refrigerator until use and was stable for at least six months.

10-15. (canceled)

16. Method of producing a radiolabelled gallium complex by reacting a Ga³⁺ radioisotope with a chelating agent characterised in that the reaction is carried out at ambient temperature.

17. Method according to claim 16, wherein the Ga³⁺ radioisotope is selected from the group consisting of ⁶⁶Ga³⁺, ⁶⁷Ga³⁺ and ⁶⁸Ga³⁺.

18. Method according to claim 16, wherein the Ga³⁺ radioisotope is ⁶⁸Ga³⁺.

19. Method according to claim 16, wherein the chelating agent is a macrocyclic chelating agent.

20. Method according to claim 16, wherein the chelating agent is a bifunctional chelating agent.

21. Method according to claim 20, wherein the bifunctional chelating agent is NOTA.

22. Method according to claim 16, wherein the chelating agent is a bifunctional chelating agent comprising a targeting vector selected from the group consisting of proteins, glycoproteins, lipoproteins, polypeptides, glycopolypeptides, lipopolypeptides, peptides, glycopeptides, lipopeptides, carbohydrates, nucleic acids, oligonucleotides or a part, a fragment, a derivative or a complex of the aforesaid compounds and small organic molecules.

23. Method according to claim 22, wherein the target vector is a peptide or oligonucleotide.

24. Method according to claim 16, wherein the reaction is carried out at 20° C. to 25° C.

25. Method according to claim 16, wherein the microwave activation is carried out for less than 10 minutes.

26. Method according to claim 18, wherein the ⁶⁸Ga³⁺ is obtained by contacting the eluate from a ⁶⁸Ge/⁶⁸Ga generator with an anion exchanger and eluting ⁶⁸Ga³⁺ from said anion exchanger.

27. Method according to claim 26, wherein the ⁶⁸Ge/⁶⁸Ga generator comprises a column comprising titanium dioxide.

28. Method according to claim 26, wherein the anion exchanger comprises HCO₃⁻ as counterions.

29. Method according to claim 26, wherein the anion exchanger is a strong anion exchanger.

30. Method according to claim 21, for the production of ⁶⁸Ga-radiolabelled PET tracers.
